(19) United States
(12) Patent Application Publication
(10) Pub. No.: US 2026/0207749 A1
(43) Pub. Date: Jul. 23, 2026

(54) NOVEL METAL OXIDE NANOPARTICLES AND COMPOSITIONS THEREOF FOR USE AS RADIOENHANCERS OR FOR VISUALIZING BIOLOGICAL TISSUE

(71) Applicant: NANOBIOTIX (NANOBIOTIX S.A), Paris (FR)

(72) Inventors: Céline Berjaud, Paris (FR); Alexandra Madeira, Paris (FR); Marie-Edith Meyre, Paris (FR); Julie Marill, Paris (FR); Pauline Morin, Paris (FR)

(73) Assignee: NANOBIOTIX (NANOBIOTIX S.A.), Paris (FR)

(21) Appl. No.: 19/137,694

(22) PCT Filed: Dec. 11, 2023
(86) PCT No.: PCT/EP2023/085150

§ 371 Date: Jun. 11, 2025

(30) Foreign Application Priority Data
Dec. 12, 2022 (EP) 22306857.8

(51) Int. Cl. A61K 41/00; A61K 9/51; A61P 35/00

(52) U.S. Cl. CPC A61K 41/0038; A61K 9/51; A61P 35/00

(57) ABSTRACT

The invention concerns inventive nanoparticles (NPs), their compositions and their use as therapeutic agents in particular in the field of oncology and/or as imaging agents. The NPs are composed of first nanoparticles of at least one high-Z metal oxide material, to which smaller second nanoparticles are covalently bonded at the first nanoparticles' surface. The second nanoparticles are composed of either a) at least one noble metal or at least one metal oxide, metal sulfide, metal selenide or metal sulfide/selenide that is a Fenton or Fenton-like reaction catalyst. The inventive NPs generate a surprisingly therapeutic effect when exposed to ionizing radiation such as X-Rays, γ-Rays, protons, neutrons, radioactive isotopes and/or electron beams. Thus, the NPs may be used as radioenhancers in the field of oncology. The nanoparticles may also be used as contrast agents.

### FIELD OF THE INVENTION

The invention concerns inventive nanoparticles (NPs), their compositions and their use as therapeutic agents, in particular, in the field of oncology and/or imaging agents. The inventive NPs generate a surprising therapeutic effect, when exposed to ionizing radiation such as X-Rays, γ-Rays, protons, neutrons, radioactive isotopes and/or electron beams. Thus, the NPs may be used as radioenhancers.

They may be used also as contrast agents for visualizing a biological tissue, preferably soft tissue, for example for diagnosis purposes, typically for detecting tumor cells in the context of oncology, for example for delineating tumor bed margins after tumor resection. The invention also relates to innovative methods for synthesizing the NPs.

### BACKGROUND

Radioenhancers are valuable in the medical field, in particular in the field of oncology. When exposed to ionizing radiation, they amplify the radiation dose deposit locally (on-off activity). When present at the cancerous tumor cell level and exposed to ionizing radiation, they augment tumor cell damage and destruction, compared to the radiation alone, and this without additional toxicity to the surrounding healthy tissues. The underlying principle of radioenhancement by high-Z nanoparticle materials is the potential of said materials to release copious amounts of electrons into a nanoscale volume, thereby amplifying radiation-induced biological damage. Thus, the primary beam (photons from, for example X-rays, or fast ions) interacts with the atoms of the high-Z radioenhancer material to generate secondary particles (photons and electrons including Auger electrons, photoelectrons, photons, k fluorescence, Compton recoil electrons, Compton scattered photons and delta electrons). In a dense medium, these secondary particles and photons successively ionize surrounding biomolecules and nanoparticles, resulting in the enhancement of the radiation effect. Radioenhancer efficacy can be measured as a “dose enhancement effect” (DEF) (Maggiorella et al. (2012) Nanoscale radiotherapy with hafnium oxide nanoparticles, Future Oncol. 8(9), 1167-1181).

While various high-Z metal-containing compositions have been identified as having radioenhancement capabilities, to date only one compound (high-density crystalline hafnium oxide nanoparticles called “NBTXR3” from the Applicant) has progressed to phase three clinical testing (for the treatment of solid tumor cancers in oncology—clinicaltrials.gov identifier NCT04892173).

Many approaches have been taken to identify radioenhancer systems that demonstrate efficacy and minimal toxic effects. For example, Metal Organic Frameworks (MOFs), Metal Organic Plates (MOPs), or Metal Organic Layers (MOLs) bearing high-Z elements have been proposed as porous radioenhancers [WO2016061256, WO2019028250]. MOFs are generally constructed by assembling metal oxo-clusters of high-Z elements using organic linkers. These porous systems carry within them small molecule drugs or other compounds for therapeutic use in vivo.

However, the latter systems present a high risk of framework degradation in vivo, as in vivo (hydrolytic) cleavage between organic linkers and the metal oxo-clusters has been reported [Kathryn E. deKrafft et al. Zr- and Hf-based nanoscale metal-organic frameworks as contrast agents for computed tomography. J Mater Chem. 2012 January 1; 22(35)].

Gold nanoclusters (AuNCs) have become a promising material for bioimaging detection because of their tunable photoluminescence, large Stokes shift, low photobleaching and good biocompatibility. U.S. Pat. No. 6,955,639 (Hainfeld et al.) describes a method of enhancing X-Rays radiation effects using metal, in particular gold, nanoparticles, the size (diameter) of the metal core being preferably, for biodistribution reasons, 0.8 to 3 nm. While literature on the potential of gold nanoparticles radioenhancers is abundant, to our knowledge, the product is not used clinically to date.

Attaining a sufficient concentration of gold atoms within the tumor while limiting the toxicity is a challenge. Chen et al. [Chen, Y. S. et al. (Assessment of the in vivo toxicity of gold nanoparticles, Nanoscale Res. Lett. 4(8) (2009) 858-64] demonstrated that, when injected intraperitoneally into BALB/C mice at a dose of 8 mg/Kg/week, gold nanoparticles ranging from 8 to 37 nm in size induced severe sickness (median survival time=21 days).

In patent application WO2013087920A1, gold nanoparticles coated with hafnium oxide were described. The aim was to protect the surrounding biological environment from gold atom-induced toxicity. No biological data was published for these compositions.

Research is active in developing new materials useful in bioimaging and/or radiation therapy. There is an unmet need to provide stable, non-toxic and efficacious products that can act as radioenhancers for use as therapeutic agents in combination with ionizing radiation. Advantageously, for example in the field of oncology, the products should also function as bioimaging agents, allowing the oncologist to visualize and treat cancerous tumors (in combination with ionizing radiation) with the same product, i.e., a “theranostic” product. These compounds must also present sustained efficacy and stability in vivo when exposed to (fractionated) radiotherapy (RT) sessions, as typically delivered in humans whatever the type of ionizing radiation used (photon, protons, electrons, etc.) and/or the radiation technique used (3D-CRT, IMRT, etc.).

Specifically, there is a need to provide materials that can be used as radioenhancers with increased efficacy compared to those in the prior art.

There is further a need to provide materials that can be used as bioimaging agents.

The inventors have identified a series of novel and inventive nanoparticles, composed of high-Z metal oxide nanoparticles, decorated with (bearing on their surface), covalently bound smaller high-Z noble metal nanoparticles and/or nanoparticles of at least one Fenton or Fenton-like reaction catalyst material selected from a metal oxide, metal hydroxide, metal oxy-hydroxide, metal peroxide, metal sulfide, metal selenide or metal sulfide/selenide. The nanoparticles are stable, non-toxic and demonstrate surprisingly high efficacy as radioenhancers. The nanoparticles may also be used as contrast agents to visualize a biological tissue, in particular soft tissue.

### SUMMARY OF THE INVENTION

It is the subject of the present invention to provide a stable radioenhancer, suitable for clinical use, with enhanced efficacy. Advantageously, the nanoparticles (“NPs”) may also be used as contrast agents to visualize, in particular, soft tissue. The nanoparticles are composed of at least one high-Z metal oxide material, on the surface of which are covalently bound, smaller noble metal nanoparticles and/or nanoparticles composed of a material that is a Fenton or Fenton-like reaction catalyst. The material that is a Fenton or Fenton-like reaction catalyst may preferably be selected from a metal oxide, metal hydroxide, metal oxy-hydroxide, metal peroxide, metal sulfide, metal selenide or metal sulfide/selenide.

Herein described is a nanoparticle (NP) or aggregate of nanoparticles (NPs), wherein the NP is composed of a first nanoparticle of at least one high-Z metal oxide (MₓO_{y}) (i.e., a metal oxide having an atomic number Z of at least 40) or mixed high-Z metal oxide (MₓM′_{z}O_{y}) (i.e., a mixed metal oxide having an atomic number Z of at least 40), the first nanoparticle having a density equal to or above (≥) 7 g/cm³ and below (<)15 g/cm³, to the surface of which smaller second nanoparticles are covalently bonded via a linker, wherein the second nanoparticles are composed of either:
a. at least one noble metal chosen from gold (Au), platinum (Pt), Ruthenium (Ru), Rhodium (Rh), palladium (Rd), osmium (Os) and iridium (Ir), and/or
b. at least one metal oxide, metal hydroxide, metal oxy-hydroxide, metal peroxide, metal sulfide, metal selenide, or metal sulfide/selenide that is a Fenton or Fenton-like reaction catalyst, wherein the metal is chosen from the group Fe, Co, Ni, Cu and Mn,
and optionally, a negatively charged or neutrally charged biocompatible surface coating, wherein the first nanoparticle metal content/second nanoparticle metal content ratio is below 30 weight/weight (w/w).

In a particular aspect, the high-Z metal oxide (MₓO_{y}) is HfO₂ or ZrO₂.

In a particular aspect, the second nanoparticles are composed of gold (Au).

In another particular aspect, the second nanoparticles are composed of iron oxide (Fe₃O₄).

In a particular aspect, the first nanoparticle metal content/second nanoparticle metal content ratio is between 20 and 30 weight/weight (w/w).

In a particular aspect, the linker on the first nanoparticle is an amino-silane and the linker on the second nanoparticle is a citrate.

In a particular aspect, the first nanoparticle has a size of between 40-100 nm.

In a particular aspect, each of the second nanoparticles has a size of between 2-15 nm.

Also herein described is a nanoparticle (NP) or an aggregate of nanoparticles (NPs) for use in altering or destroying target cells in a mammal when said cells are exposed to ionizing radiation chosen from X-rays and protons. In a particular aspect, cells are exposed to ionizing radiation in the context of a radiotherapy regimen selected from a conventional fractionation regimen, a hyperfractionation regimen, a(n) (accelerated) hypofractionation regimen, and a Stereo Ablative Body radiotherapy (SBAR) regimen. In another particular aspect, the target cells are from a solid malignant tumor selected from a skin cancer, a central nervous system cancer, a head and neck cancer, a lung cancer, a breast cancer, a gastrointestinal cancer, a male genitourinary cancer, a gynecologic cancer, an adrenal and/or retroperitoneal cancer, a sarcoma and a pediatric cancer.

Also herein described is a nanoparticle (NP) or aggregates of nanoparticles (NPs) for use in visualizing biological tissue, in particular soft tissue, in a mammal, thereby enabling the detection of tumor cells and/or diagnosis of cancer.

Further herein described is a composition comprising a) nanoparticles (NPs) or aggregates (NPs) of the invention, or their mixtures, and b) a pharmaceutically acceptable carrier, vehicle or support.

A particular composition of the invention is for use in altering or destroying target cells, in particular tumor cells, in a mammal, particularly in a human, when said cells are exposed to ionizing radiation.

Another particular composition of the invention is for use in visualizing biological tissue, in particular soft tissue, in a mammal, using CT scan, thereby enabling the detection of tumor cells and/or diagnosis of cancer.

In one embodiment of the invention, the composition comprising the NPs or aggregates thereof is for use in altering or destroying target cells, in particular tumor cells, in a mammal, particularly in a human, when said cells are exposed to ionizing radiation.

### FIGURES

FIG. 1.A Transmission Electronic Microscopy (TEM) image of Example 2 (scale bar=50 nm), JEOL2100Plus was used for image analysis.

FIG. 1.B Transmission Electronic Microscopy (TEM) image of Example 3.B. The presence of gold nanoparticles on HfO₂ nanoparticles (scale bar=20 nm) is confirmed. JEOL 2100Plus was used for image analysis.

FIG. 1.C Transmission Electronic Microscopy (TEM) image of Example 3.B. The presence of gold nanoparticles on HfO₂ nanoparticles was confirmed (scale bar=100 nm). JEOL 2100Plus was used for image analysis.

FIG. 2 The Surviving Fraction values after irradiation of 4 Gy from the mean of at least three independent experiments for Examples 1, 3.B (800 μM) and Example 2 (32 μM) activated by X-ray in CT26 cells. Values are expressed as SF mean±SD (n≥3) at 4 Gy. * indicates a p-value <0.05.

FIG. 3.A Transmission Electronic Microscopy (TEM) image of Example 5 (scale bar=50 nm), JEOL 2100Plus was used for image analysis.

FIG. 3.B and C. Transmission Electronic Microscopy (TEM) image of Example 6. The presence of gold nanoparticles on surface of the HfO₂ nanoparticles confirmed. FIG. 3.B: scale bar=20 nm, FIG. 3.C: scale bar=100 nm. JEOL 2100Plus was used for image analysis.

### DEFINITIONS

A high-Z (metal) element is an element of Mendeleev's periodic table that has an atomic number value (i.e. a Z value) of at least 40, preferably above 45, 50, 55, 60, 61, 62, 63, 64, 65, 66, 67, 68, 69, 70, 71, 72, 73, 74, 75, 76, 77, 78, 79, 80 or 81. In the context of the present invention, the high-Z metal oxide is an oxide of a high-Z metal (MₓO_{y}) or a mixed metal oxide MₓM′_{z}O_{y} of a high-Z metal wherein M and M′ are metal elements and M or M′ has a Z value of at least 40. Typically, M or M′ can be a lanthanide element, a zirconium (Zr) element, or a hafnium (Hf) element.

Noble Metal is defined herein as a metal selected from gold (Au), platinum (Pt), Ruthenium (Ru), Rhodium (Rh), palladium (Rd), osmium (Os) and iridium (Ir), in particular selected from gold (Au), platinum (Pt), Ruthenium (Ru), Rhodium (Rh), palladium (Rd), osmium (Os) and iridium (Ir).

The term “crystallite” herein refers to a crystalline product. The size of the crystallite and its structure and composition may be analyzed from an X-ray diffractogram.

The term “aggregate of nanoparticles” refers to an assemblage of nanoparticles strongly, typically covalently, bound to each other. Similarly, the term “aggregate of crystallites” refers to an assemblage of crystallites strongly, typically covalently, bound to each other. In the context of the invention, the inventive nanoparticles (NPs) may be in the form of nanoparticles or aggregates of nanoparticles. The inventive NPs themselves are each composed of a first nanoparticle decorated with (smaller) second nanoparticles. To form the inventive NPs, it is preferable that the first and second nanoparticles are not themselves in the form of aggregates. It is preferred that the first and second nanoparticles have sufficient colloidal stability to ensure that aggregation does not occur. The skilled person understands that colloidal stability of nanoparticles suspensions may be largely influenced by nanoparticle surface charge potential, or zeta potential. Reference may be made to Norm ASTM E 2865-2012 for information about the relationship between surface charge and colloidal stability. Aggregation of the first and/or second nanoparticles may prevent second nanoparticles bonding to the surface of first nanoparticles to form the reactive NPs.

The terms “treatment” or “therapy” refer to both therapeutic and prophylactic or preventive treatment or measures that can significantly slow disease progression (for example, stop cancerous tumor growth) or increase/improve Progression Free Survival (PFS) or Overall Survival (OS), or cure cancer (i.e., turn the patient into a cancer survivor, as further defined herein below).

Such a treatment or therapy is intended for a subject in need thereof, in particular, a mammal, preferably a human being, typically a human patient suffering from a malignant solid tumor.

In the art and the context of the present invention, the terms “treatment having curative intent”, “curative treatment” or “curative therapy” refer to treatment or therapy, in particular, a treatment comprising a radiotherapeutic step, offering to the subject to be treated a curative solution for treating cancer(s) he/she is affected by, that is, for globally treating said subject [primary tumor(s) as well as a corresponding metastatic lesion(s)].

As well known by the skilled person, the terms “palliative treatment” including in particular “palliative radiotherapy” are used for palliation of symptoms and are distinct from “radiotherapy”, i.e., radiotherapy delivered as curative treatment (also herein identified as “curative radiotherapy”).

Indeed, palliative treatment is considered by the skilled person as an efficacious treatment for treating many symptoms induced by locally advanced or metastatic tumors, even for patients with short life expectancy.

“Fenton reaction” is the reaction reported by Fenton in 1894, who found that Fe²⁺ catalyzed tartaric acid oxidation in H₂O₂ containing solution. The general reaction mechanism of Fenton reaction (shown in equations (1) and (2) below), was put forward by Haber and Weiss in 1934:

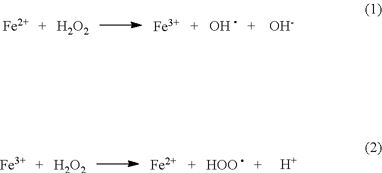

«Fenton-like reaction» is known as a Fenton reaction carried out using a transition metal ion that is not iron, for example, Cu²⁺, Mn²⁺ and CO²⁺. The latter metal ions act as catalysts to produce OHs.

To avoid confusion, the abbreviation of the word nanoparticle, “NP” or “NPs”, is used herein only for the inventive nanoparticle(s) (or aggregate(s) thereof), composed of a first nanoparticle of at least one high-Z metal oxide material, to which smaller second nanoparticles are covalently bound at the first nanoparticles surface, the second nanoparticles being composed of at least one noble metal, and/or at least one Fenton or Fenton-like reaction catalyst material. “NP” is not used as an abbreviation for the first or second nanoparticles taken separately.

### DETAILED DESCRIPTION OF THE INVENTION

Inventors herein describe various aspects of the invention. The inventive nanoparticles (NPs) are composed of first nanoparticles onto the surface of which second nanoparticles are bonded. Thus, the NPs are composed of first nanoparticles decorated with second nanoparticles.

### First Nanoparticle of High-Density High-Z Metal Oxide

The first nanoparticles are composed of a high-Z metal oxide material. The metal oxide of interest herein described may be selected from the group consisting of an oxide from a lanthanide element and an oxide from a chemical (metallic) element of the periodic classification of elements (Mendeleev's table), in particular an oxide from a metallic element with Z equal to or above 40. The first nanoparticle has a density equal to or above (≥) 7 g/cm³ and below (<) 15 g/cm³. Density is mass m per unit volume V. It has been shown in patent application WO2009/147214 that improved therapeutic efficacy is obtained using nanoparticles having this density range. Thus, the first nanoparticle may have a density of at least 7.5 g/cm³, preferably at least 8 g/cm³, even more preferably of at least 8.5, 9, 9.5, 10, 10.5, 11, 11.5, 12, 12.5, 13, 13.5 or 14 g/cm³. The density of the nanoparticles or nanoparticles' aggregates may be determined from approximately 1 g of dried powder using Accupyc 1340 pycnometer equipment. Such nanoparticles are described in patent application WO2009/147214.

Examples of suitable (high-Z) metal oxides herein described are:
appropriate oxides from a lanthanide element, which may be selected for example from the group consisting of Nd₂O₃, Sm₂O₃, Eu₂O₃, Gd₂O₃, Tb₂O₃, Dy₂O₃, Ho₂O₃, Er₂O₃, Tm₂O₃, Vb₂O₃, Lu₂O₃ and any mixtures thereof;
appropriate oxides from a metallic element of period 6 of the periodic classification of elements, which may be selected, for example, from the group consisting of HfO₂, TaO₂, Ta₂O₅, WO₂, WO₃, ReO₂, OsO₂, IrO₂, PtO, PtO₂, HgO, Hg₂O, Tl₂O₃, Bi₂O₃ and any mixtures thereof;
appropriate oxides from a metallic element of period 5 of the periodic classification of elements, which may be selected for example from the group consisting of NbO, RuO₂, Rh₂O₃, RhO₂, PdO, Ag₂O, AgO, CdO, In₂O₃, and any mixtures thereof.

Preferred nanoparticles are made of HfO₂, ZrO₂, PdO, or ReO₂, or mixtures thereof. Especially preferred nanoparticle materials are HfO₂ and ZrO₂. Reference is made to WO 2009/147214 for further description of the synthesis of HfO₂ nanoparticles and the adjustment of synthesis parameters to obtain desirable nanoparticle, or nanoparticle aggregate, density. The first nanoparticles are generally crystalline.

The synthesis for HfO₂ first nanoparticles, according to one embodiment of the invention, is described in Example 3A hereinbelow. Of note, the chemistry of zirconium cation and hafnium cation in an aqueous solution is similar [C. F. Baes and R. S. Mesmer: The Hydrolysis of Cations. John Wiley & Sons, New York, London, Sydney, Toronto 1976]. Therefore, the zirconium cations can easily substitute the hafnium cations in the herein below described syntheses.

### Size:

In the spirit of the invention, the term “nanoparticle” refers to a product, in particular a synthetic product, with a size typically between about 1 nm and about 1000 nm, preferably between about 1 nm and about 500 nm.

The size of the particle can typically be measured by Electron Microscopy (EM) techniques, such as transmission electron microscopy (TEM) or cryo-TEM, as well known by the skilled person. The size of at least 100 particles is typically measured (typically considering the particle's longest dimension) and the median size of the population of particles is reported as the size of the particles.

The (first) high-Z metal oxide nanoparticle has a size in the range of 20-150 nm, preferably 30-120 nm, more preferably 40-100 nm. As stated above, the nanoparticles may be in the form of crystallites or aggregates of crystallites.

The inventive NP composed of the first high-Z metal oxide nanoparticle with the second nanoparticles bound at its surface may have a size in the range of 20-500 nm, preferably 40-200 nm, more preferably 40-120 nm, even more preferably 40 to 100 nm. Similarly, the NPs may be in the form of crystallites or aggregates of crystallites.

A second nanoparticle typically has a size in the range of 2-15 nm, preferably 3-10 nm, more preferably 3-6 nm.

As the shape of the particles can influence their biocompatibility, particles having a quite homogeneous shape are preferred. For pharmacokinetic reasons, particles being essentially spherical, round, or ovoid in shape are, thus preferred. Such a shape also favors the particles' interaction with or uptake by cells.

### Second Metal Nanoparticle

As described above, the inventive NPs are composed of a first nanoparticle of at least one high-Z metal oxide material (MₓO_{y}) or mixed high-Z metal oxide (MₓM′_{z}O_{y}), on the surface of which are covalently bound smaller noble metal nanoparticles and/or nanoparticles of a material capable of catalyzing a Fenton or Fenton-like reaction, typically a metal oxide, metal hydroxide, metal oxy-hydroxide, metal peroxide, metal sulfide, a metal selenide or metal sulfide/selenide. The first nanoparticles composed of a high-Z metal oxide material or mixed high-Z metal oxide typically have a density of between 7 and 15 g/cm³, preferably equal to or above (≥) 7 g/cm³ and below (<) 15 g/cm³.

According to one embodiment of the invention, the (second) nanoparticles covalently bound at the first nanoparticles' surface are of at least one noble metal.

According to one embodiment of the invention, the (second) nanoparticles covalently bound at the first nanoparticles' surface are of at least one Fenton or Fenton-like reaction catalyst material. The Fenton or Fenton-like reaction catalyst material may be selected from at least one metal oxide, metal hydroxide, metal oxy-hydroxide, metal peroxide, metal sulfide, metal selenide or metal sulfide/selenide.

According to one embodiment of the invention, (second) nanoparticles covalently bound at the first nanoparticles' surface are of a mixture of nanoparticles of at least one noble metal and nanoparticles of at least one Fenton and/or Fenton-like reaction catalyst material. Said material may be at least one metal oxide, metal hydroxide, metal oxy-hydroxide, metal peroxide, metal sulfide, metal selenide or metal sulfide/selenide capable of catalyzing a Fenton or Fenton-like reaction.

### Nanoparticles of at Least One Noble Metal Material:

Generally, the nanoparticle noble metal material can be gold (Au), platinum (Pt), Ruthenium (Ru), Rhodium (Rh), palladium (Rd), osmium (Os) or iridium (Ir), or any mixture thereof. A mixture of noble metals is known as an alloy. For example, the noble metal material may be an alloy of gold and platinum, or of gold and palladium, or of palladium and platinum. According to one embodiment of the invention, a preferred noble metal material is gold, platinum or palladium. A particularly preferred noble metal material is gold.

Any of the methods known to the skilled person may be used for the synthesis of noble metal nanoparticles. The synthesis for gold nanoparticles, according to one embodiment of the invention is described in Example 2 hereinbelow.

The size of the noble metal nanoparticle is in the range of 2-15 nm, preferably 3-10 nm, more preferably 3-6 nm.

For pharmacokinetic reasons, particles being essentially spherical, round, or ovoid in shape are, thus preferred.

High-Z Metal Oxide Electronic Cooperation with Noble Metal Nanoparticle Surface Plasmons:

Noble metal nanostructures display unique and strongly enhanced optical properties due to the phenomenon of localized surface plasmon resonance (LSPR). In assemblies or complex noble metal nanostructures, individual plasmon oscillations on proximal particles can couple via their near-field interaction, resulting in coupled plasmon resonance modes, quite akin to excitonic coupling in molecular aggregates or orbital hybridization in molecules [Jain and EL-Sayed (2010) “Plasmonic coupling in noble metal nanostructures” Chem. Phys. Lett., vol 487, pp 152-164]. Hence, the plasmon in a noble metal nanoparticle can be considered as a mass-spring harmonic oscillator driven by the energy resonant light wave, where the electron cloud oscillates like a simple dipole in parallel direction to the electric field of the electromagnetic radiation [Amendola et al., (2017) Surface plasmon resonance in gold nanoparticles: a review. J. Phys.: Condens. Matter 29 203002 (pp. 48)].

Without being bound by theory, the inventors assume that after irradiation with ionizing radiation (i) the yield of the low energy electrons (LEE), like Auger electrons, emitted by the high-Z metal oxide nanoparticle and/or (ii) the LEE trajectory distance, is/are enhanced by the noble metal nanoparticle surface plasmon. Indeed, both Auger electrons and surface plasmon electrons are confined at the nanoparticle surface and Auger electrons present a very short range, which is less than 10 nm (Kuncic and Lacombe, (2018) Nanoparticle radio-enhancement: principles, progress and application to cancer treatment. Phys. Med. Biol. 63, 02TR01 (27 pp)). The interaction between the noble metal surface plasmon and LEEs, such as Auger electrons, emitted by the metal oxide nanoparticle after irradiation is facilitated by the short distance (typically less than about 40 Å) between the first and second nanoparticle surfaces, in particular, the first and second nanoparticles are bonded covalently via a relatively short linker (described below).

According to one embodiment of the invention, the second nanoparticles of at least one noble metal are covalently bound to the surface of each first nanoparticle. According to one embodiment of the invention, at least 80% or 85%, more preferably at least 90% or 95% of the noble metal nanoparticle surface is exposed to the surface so that the noble metal nanoparticle surface plasmons yield is maximized. In other words, according to an embodiment of the invention, less than 20% or 15%, more preferably less than 10% or 5% of noble metal nanoparticle surface is embedded in the first nanoparticle (and therefore less accessible to LEEs emitted from the high-Z metal oxide nanoparticles once irradiated with ionizing radiation).

The surface area of the nanoparticles of the invention (NPs) may be determined by BET (Brunauer, Emmett and Teller) surface area analysis or by cetyltrimethylammonium bromide (CTAB) surface area analysis. The CTAB surface area analysis is preferred. The CTAB surface area analysis is based on the absorption of the cetyltrimethylammonium bromide (CTAB) molecule on the surface of the nanoparticles (the nanoparticles are in solution). The CTAB molecule is relatively large so that it is not adsorbed in micropores. Thus, the CTAB surface area reflects only the surface of the nanoparticle that is available for interaction with ionizing radiation. Other molecules (such as proteins) could be used otherwise in the context of the invention to estimate this nanoparticle surface area.

### Second Nanoparticle Composed of a Fenton Reaction Catalyst Material or Fenton-Like Reaction Catalyst Material

According to one embodiment of the invention, the second nanoparticles may be composed of a metal oxide, metal hydroxide, metal oxy-hydroxide, metal peroxide, metal sulfide, metal selenide or metal sulfide/selenide that acts as a Fenton reaction catalyst or a Fenton-like reaction catalyst. Generally, Fenton/Fenton-like reactions triggered by the tumor microenvironment (TME) are regarded to be the most representative of chemodynamic therapy (CDT). Generally, they use transition metal ions (e.g., Fe, Co, Ni, Cu and Mn) to catalyze the decomposition of hydrogen peroxide (H₂O₂) with low activity and produce high-toxicity hydroxyl radicals (·OH). The hydroxyl radicals cause damage to the cellular DNA resulting in cell damage and/or death.

According to one embodiment of the invention, the second nanoparticles are composed of a metal oxide, metal hydroxide, metal oxy-hydroxide, or metal peroxide of Fe, Co, Ni, Cu or Mn. According to one embodiment of the invention, the second nanoparticles are composed of at least one mixed metal oxide of Fe, Co, Ni, Cu or Mn.

According to one embodiment of the invention, the second nanoparticle Fenton reaction/Fenton-like reaction catalysts are composed of at least one iron oxide such as magnetite (Fe₃O₄), maghemite (γ-Fe₂O₃) or hematite (α-Fe₂O₃), an iron oxy-hydroxide like goethite (FeOOH), a copper oxide (CuO), a copper peroxide (CuO₂), a manganese oxide (MnO), a cobalt oxide (CoO), a nickel oxide (NiO) or any mixture thereof.

According to a preferred embodiment, the second nanoparticle is composed of magnetite, also written as iron (II,III) oxide Fe₃O₄. It contains both Fe²⁺ and Fe³⁺ ions and may be formulated as FeOFe₂O₃.

Suitable methods for the synthesis of these metal salt nanoparticles are known to the skilled person. The synthesis for iron (II, III) oxide Fe₃O₄ nanoparticles according to one embodiment of the invention is described in Example 5A herein below.

According to one embodiment of the invention, the second nanoparticles are composed of a metal sulfide that acts as a Fenton reaction catalyst or a Fenton-like reaction catalyst. According to one embodiment of the invention, the second nanoparticles are composed of FeS, FeS₂, Fe₃S₄, CuS, Cu₂S, MnS, NiS₂, Ni₃S₂, CoS, CoS₂, Co₃S₄, Co₉S₈ or any mixture thereof. Preferably, the second nanoparticles are composed of CuS, Cu₂S, MnS, FeS, FeS₂ or any mixture thereof. More preferably, the second nanoparticles are composed of FeS, FeS₂, Fe₃S₄ or any mixture thereof.

According to one embodiment of the invention, the second nanoparticles are composed of a metal selenide. According to one embodiment of the invention, the second nanoparticles are composed of CuSe, Cu₂Se, FeSe, FeSe₂, Fe₃Se₄, MnSe, NiSe, NiSe₂, Ni₂Se₃, CoSe, CoSe₂, Co₂Se₄, or any mixture thereof. Preferably, the second nanoparticles are composed of CuSe, Cu₂Se, MnSe, FeSe, FeSe₂, Fe₃Se₄ or any mixture thereof. More preferably, the second nanoparticles are composed of FeSe, FeSe₂, Fe₃Se₄ or any mixture thereof.

According to one embodiment of the invention, the second nanoparticle of Fenton reaction/Fenton-like reaction catalyst material are composed of a mixture of metal sulfide and metal selenide (herein also identified as “metal sulfide/selenide” or “sulfide/selenide” catalysts). In the latter class of compounds, Se atoms may substitute some of the sulphur atoms in the metal sulfur structure. According to one embodiment of the invention, the second nanoparticles are composed of FeSSe, CuₓS/CuₓSe wherein X is equal to 1 or 2, or mixed sulfide/selenide compounds of Cobalt and/or Nickel.

The size of the second nanoparticles composed of a material that acts as a Fenton reaction catalyst or a Fenton like reaction catalyst is generally in the range of 2-15 nm, preferably 3-10 nm, more preferably 3-6 nm.

The second nanoparticles may be spherical, round, ovoid or cubic in shape. The second nanoparticles may be nanocubes.

For pharmacokinetic reasons, particles being essentially spherical, round, or ovoid in shape are, thus preferred.

### Electronic Cooperation Between High-Z Metal Oxide Nanoparticles and Fenton or Fenton-Like Reaction Catalyst Nanoparticles:

Without being bound by theory, it is thought that the efficacy of the inventive NPs may be due to cooperation between electronic systems. Specifically, electrons produced by the interaction of the first nanoparticle high-Z metal oxide nanoparticles with ionizing radiation create an electronic environment (including emitted photoelectrons, and LEE electrons such as Auger electrons) favorable for reducing Fe³⁺ to Fe²⁺. Subsequently, Fe²⁺ can react with H₂O₂ to produce OH· radicals responsible for DNA damage in the cancerous tumor cells. Thus, when a mix of Fe²⁺/Fe³⁺ ions is present at the surface of the high-Z metal oxide (for example, HfO₂) nanoparticles, a higher amount of Fe²⁺ ions may be generated, due to the electron rich environment provided by the irradiated high-Z nanoparticle. This may lead to a higher production of OH· radicals and to a local increase of OH· radicals concentration (dose increase).

According to one embodiment of the invention, Fe₃O₄ nanoparticles are covalently bound to HfO₂ nanoparticles. Fe₃O₄ contains both Fe²⁺ and Fe³⁺ (Fe²⁺ Fe³⁺₂ O₄). Such an electronic environment is favorable to Fe²⁺ production. In addition, as exposed in the state of the art, the use of Fe₃O₄ (giving a global oxidation state of +2.5) has been proven very efficient for the decomposition of H₂O₂ into OH· radicals [Xue, X. et al (2009). Journal of hazardous materials, 166(1), 407-414)].

### Linker

Generally, the second nanoparticles are covalently bound to the surface of the first nanoparticles via a linker group. The linker group may be formed by the reaction of functional groups on the first and second nanoparticles' surfaces. Thus, the first nanoparticles may be functionalized with a chemical group that can react with a chemical group with which the second nanoparticles are functionalized. In the context of the invention the term “functionalized with” means strongly bonded with a functional group, typically by covalent bonding. For example, the first nanoparticles may be functionalized with an amine group according to methods known to the skilled person. In that case, the second nanoparticles may thus be functionalized with a functional group that reacts with an amine group, for example a citrate, cysteine or dopamine leading to the formation of a linker. The thus formed linker typically has, preferably, a maximum thirty (30) bonds length between both particles, thereby ensuring that electronic cooperation may be facilitated between the two nanoparticles. One may cite as an example an amide linker. Other linkers may be used, for example a disulfide group containing linker or a triazole linker.

Table 1 below shows a non-exhaustive list of examples of reactive groups and suitable agents that may be used to graft (chemically bind) reactive groups onto a first nanoparticle of the invention.

TABLE 1
linkers for first nanoparticles

| Reactive groups grafted on | |
|---|---|
| first nanoparticles | Examples of reagent |
| Primary amine | Amino silane |
| | with 3 < n_{CH2} < 12 |
| Thiol | Amino silane + 2-iminothiolane |
| Carboxylic acid | Silane-PEG-carboxylic acid |
| Alkyne | Alkyne-PEG-acid |
| Azide | Silane-PEG-Azide |

Table 2 below shows a non-exhaustive list of examples of reactive groups and suitable agents that may be used to bond reactive groups onto a second nanoparticle. Linker groups are indicated according to the type of second nanoparticle used.

TABLE 2
linkers for second nanoparticles

| | | Noble | Metal |
|---|---|---|---|
| Reactive groups | Examples of reagent | metal | oxide |
| Carboxylic acid | Citrate | X | |
| Thiol | Butanedithiol | X | |
| Primary amine | Amino thiol with 3 < n_{CH2} < 12 | X | |
| Primary amine | Amino silane with 3 < n_{CH2} < 12 | | X |
| | Dopamine | | |
| Carboxylic acid | 3-(Triethoxysilyl)propyl | | X |
| | succinid acid | | |
| Azide | Thiol-PEG-Azide | X | X |
| Azide | 3-Azidopropionic acid | | X |
| Alkyne | Alkyne-PEG-acid | | X |
| Alkyne | Alkyne-PEG-Thiol | X | |

Table 3 below indicates non-exhaustive examples of strategies that may be used to create a covalent bond between functionalised second particles and first nanoparticles.

TABLE 3
Suitable linker activators to create a covalent bond between
functionalised second particles and first nanoparticles

| Reactive groups | | Reactive groups | |
|---|---|---|---|
| bonded to first | Type of linker/ | bonded to second | Type of |
| nanoparticles | activator | nanoparticles | Bond |
| —NH₂ | EDAC with or without | —COOH | Amide |
| | Hydroxysulfosuccinimide | | |
| —COOH | EDAC with or without | —NH₂ | Amide |
| | Hydroxysulfosuccinimide | | |
| —SH | / | —COOH | Ligand |
| | | | exchange |
| —SH | X—R—X, where X = | —SH | Thioether |
| | maleimide | | |
| —N═N═N | Copper(I)-catalyzed | —C≡C | Triazole |
| | Huisgen cycloaddition | | |
| —C≡C | Copper(I)-catalyzed | —N═N═N | Triazole |
| | Huisgen cycloaddition | | |

### Ratio of First/Second Nanoparticle Metal Content

The first nanoparticle metal content/second nanoparticle metal content ratio is generally less than 35 weight/weight (w/w), preferably less than 30 (w/w), and more preferably less than 25 (w/w). A first nanoparticle metal content/second nanoparticle metal content ratio between 20 and 30 w/w is preferred.

Indeed, the inventors have identified the ratio of 20-30 based on the excellent efficacy results obtained herein and discussed below. The NPs have also shown to be non-toxic. The inventors observed in previous experiments that nanoparticles with Hf/Au ratio outside of this range are less efficient. In Reference Example 7 below, a product obtained by electrostatic bonding between first and second nanoparticles was synthesized. The product had a Hf/Au ratio of 86, a ratio too high to obtain the synergistic electronic effect seen for the inventive NPs described herein. These experiments helped to define a target first nanoparticle metal content/second nanoparticle metal content ratio (w/w) as being preferably below 30.

The respective metal content of the nanoparticles may be measured using methods known to the skilled person. For example, the nanoparticle metal content may be determined using Inductively Coupled Plasma-Optical Emission Spectrometry (ICP-OES). According to one embodiment of the invention, wherein the first nanoparticle is hafnium oxide (HfO₂) and the second nanoparticle is gold (Au) the ratio Hf/Au may be in the range 20-30 (w/w), preferably in the range 25-30 (w/w), for example of 25, 26, 27, 28, 29 or 30 (w/w).

According to one embodiment of the invention, wherein the first nanoparticle is hafnium oxide (HfO₂) and the second nanoparticle is iron oxide (Fe₃O₄), the ratio of Hf/Fe is in the range 20-30 (w/w) preferably in the range 25-30 (w/w), for example of 25, 26, 27, 28, 29 or 30 (w/w).

The inventors have found that NPs having a ratio of first nanoparticle metal content/second nanoparticle metal content that falls within the range 20 to 30, preferably 25 to 30, have greater than expected radioenhancer capability, based on the measured radioenhancer capability of the first nanoparticles and second nanoparticles, each taken separately This synergistic or cooperative radioenhancer effect has been demonstrated in the results of the Applicant's experiments. DEF for the inventive NPs is greater than the combined effect of the first (1°) and second (2°) nanoparticles taken separately. The effect may be expressed as the following:

$${DEF}_{inventive NP} > {DEF}_{1 ° nanoparticle} \times {DEF}_{2 ° nanoparticle}$$

According to one embodiment of the invention, the first nanoparticles are hafnium oxide first nanoparticles and the second nanoparticles are gold second nanoparticles. Example 3B below is an example of such an embodiment. The radioenhancer effect of the latter NP was tested in a clonogenic assay (see Example 4 below). The data in Example 4 below indicates that the DEF of the (first hafnium oxide nanoparticles was 4.49 (±0.266), and the DEF of the (second) gold nanoparticles was measured to be 1.16 (±0.282). The DEF of the inventive NPs (hafnium oxide nanoparticles with gold nanoparticles covalently bound to the surface of hafnium oxide nanoparticles and having a hafnium/gold ratio (w/w) of 27 was measured to be 7.88 (±0.736). 7.88 (±0.736) is greater than the combined DEF of the first and second nanoparticles taken separately (1.16 multiplied by 4.49=5.21). Using the error margins, to take the highest DEF values for the first nanoparticles and second nanoparticles, and to take the lowest DEF value for the inventive nanoparticles (NPs), the cooperative effect is still seen, i.e., (4.49+0.266)×(1.16+0.282)=6.85 and 7.88-0.746=7.13. 7.13>6.85.

The inventors have thus identified a specific range of second nanoparticles that allows the electronic “cooperation” with the electrons emitted after irradiation of the first metal oxide nanoparticle. Furthermore, NPs with a metal/metal (w/w) ratio in the range 20-30, show no additional toxicity compared to the first nanoparticles alone.

### Optional Biocompatible Coating

In a particular aspect of the description, each NP composed of the first and second nanoparticles of the present invention is coated with a biocompatible surface coating.

In a preferred aspect, each NP used in the context of the present invention is coated with a biocompatible material, preferably with an agent exhibiting a stealth property. Indeed, when the NPs of the present invention are administered to a subject via the intravenous (IV) route, a biocompatible coating with an agent exhibiting a stealth property is particularly advantageous to optimize the biodistribution of the NPs. Such coating is responsible for the so called “stealth property” of the NPs. The agent exhibiting stealth properties may be an agent displaying steric hindrance at the NPs surface. Such an agent may be selected for example from polyethylene glycol (PEG); polyethylene oxide; polyvinylalcohol; polyacrylate; polyacrylamide (poly(N-isopropylacrylamide)); polycarbamide; a biopolymer; a polysaccharide such as for example dextran, xylan and cellulose; collagen; and a zwitterionic compound such as for example polysulfobetain.

In another preferred aspect, the NPs can be coated with an agent allowing non-specific interaction with a biological target. Such an agent can typically bring a negative or a neutral charge on the NP surface. This charge can be easily determined by zeta potential measurements, typically performed on NP suspensions the concentration of which vary between 0.2 and 10 g/L, the particles being suspended in an aqueous medium with a pH comprised between 6 and 8.

An agent forming a negative charge on the NPs surface can be for example a phosphate (for example, a polyphosphate, a metaphosphate, a pyrophosphate, etc.), a carboxylate (for example, a citrate or dicarboxylic acid, in particular a succinic acid) or a sulphate. Polyphosphates are preferred. Hexametaphosphate is particularly preferred.

A full biocompatible coating of the NPs may be advantageous, in particular for an intravenous (IV) administration in the human patient, in order to avoid interaction of the particle's surface with any recognition element of the immune system (macrophage, opsonins, etc.) The “full coating” implies the presence of a very high density of biocompatible molecules able to create at least a complete monolayer on the surface of the particle.

The biocompatible coating allows, in particular, the nanoparticle stability between pH 6.5 and 7.5 in a biocompatible suspension, such as a physiological fluid (blood, plasma, serum, etc.), any isotonic media or physiologic medium, for example media comprising glucose (5%) and/or NaCl (0.9%) required for a pharmaceutical administration.

Such a biocompatible coating is obtained by treating the nanoparticle with a surface treating agent. Stability may be confirmed by dry extract quantification measured on a nanoparticle suspension prior and after filtration on a 0.22 pm filter.

Advantageously, the coating ensures or improves the biocompatibility of the particles in vivo, and facilitates an optional functionalization thereof (for example with spacer molecules, biocompatible polymers, targeting agents, proteins, etc.).

### Optional Targeting

The NPs can further comprise a targeting agent. A targeting agent typically recognizes an element present on a target cell, typically on a cancer cell. Such a targeting agent typically acts once the NPs of the invention are accumulated on the target site, typically on the tumor site. The targeting agent can be any biological or chemical structure displaying affinity for molecules present in the mammalian, in particular the human, body. For instance, it can be a peptide, oligopeptide or polypeptide, a protein, a nucleic acid (DNA, RNA, SiRNA, tRNA, miRNA, etc.), a hormone, a vitamin, an enzyme, the ligand of a molecule expressed by a pathological cell, in particular the ligand of a tumor antigen, hormone receptor, cytokine receptor or growth factor receptor. Said targeting agent can be for example selected in the group consisting in LHRH, EGF, a folate, anti-B-FN antibody, E-selectin/P-selectin, anti-IL-2Ra antibody and GHRH.

### Administration Route

The NPs of the invention can be administered to the subject using different possible routes such as local (for example, intra-tumoral (IT)) or systemic (for example, intra-arterial (IA)) administration. The administration may be, for example a subcutaneous administration, intra-dermic administration, intra-arterial (IA) administration, intravenous (IV) administration, airways administration (inhalation), intraperitoneal administration, intramuscular administration, intra-articular administration, intra-thecal administration, intra-ocular administration or oral route administration (per os). Preferably the administration is performed through the IT, IV or IA route. The intratumoral (IT) route is the most preferred.

Repeated injections or administrations of NPs can be performed, when appropriate.

### Cancer Indications for Treatment

The NPs herein described are typically for use in altering or destroying target cells in a mammal, in particular, a human, when said cells are exposed to ionizing radiation.

The herein described invention can be applied to any target cells, in particular tumor cells, typically in a human patient suffering from a cancer.

The cancer is for example a skin cancer, in particular a malignant neoplasm associated to AIDS; a melanoma; a squamous cancer; a central nervous system cancer such as for example a brain, cerebellum, pituitary, spinal cord, brainstem, eye or orbit cancer; a head and neck cancer, a lung cancer, a breast cancer, a gastrointestinal cancer such as a liver or a hepatobiliary tract cancer, a colon, a rectum and/or an anal cancer, a stomach cancer, a pancreas cancer, an esophagus cancer; a bladder or urethra cancer, a male genitourinary cancer such as for example a prostate, testis, or penis cancer; a gynecologic cancer, such as for example, a uterine cervix, endometrium, ovary, fallopian tube, vagina and/or vulvar cancer; an adrenal and/or retroperitoneal cancer; a sarcoma of bone and soft tissue regardless its localization; or a pediatric cancer such as for example a Wilm's cancer, a neuroblastoma, a central nervous system cancer or a Ewing's sarcoma.

In a particular aspect of the invention, the target cells belong to a solid malignant tumor selected from a skin cancer, a central nervous system cancer, a head and neck cancer, a lung cancer, a breast cancer, a gastrointestinal cancer, a male genitourinary cancer, a gynecologic cancer, an adrenal and/or retroperitoneal cancer, a sarcoma and a pediatric cancer.

### Radiotherapy Sources

In a typical aspect herein described, the inventive NPs are to be administered to the subject to be treated and said subject is then to be exposed to ionizing radiation. This ionizing radiation is typically selected from X-rays, gamma-rays (γ-Rays), protons, neutrons, radioactive isotopes and/or electrons. Preferred ionizing radiation is X-rays and protons. Even more preferred ionizing radiation is X-rays.

In a preferred aspect, the NPs of the invention, or the subject who has been administrated with the NPs of the invention, is to be exposed to ionizing radiation.

RT comprises multiple different treatment modalities, including external beam therapy (encompassing photons, electrons, protons and other particles) and internal/surface treatment (brachytherapy and radiopharmaceuticals). In a preferred aspect herein described, ionizing radiation is selected from X-rays, gamma-rays, electrons and protons. In a preferred aspect herein described, ionizing radiation is chosen from X-rays and protons.

As indicated herein above, appropriate radiation is preferably ionizing radiation and can advantageously be selected from the group consisting of X-Rays, gamma-Rays, electron beams (electrons), ion beams (such as protons) and radioactive isotopes or radioisotopes emissions. X-Rays are particularly preferred ionizing radiation.

Ionizing radiation are those of standard of care. They are typically of about 2 KeV to about 25 000 KeV, in particular of about 2 KeV to about 6000 KeV (i.e. 6 MeV) (LINAC source).

In general, and in a non-restrictive manner, the following X-Rays can be applied in different circumstances to excite the herein described nanoparticles:
Superficial X-Rays of 2 to 50 keV: to excite nanoparticles near the skin surface (penetration of a few millimeters);
X-Rays of 50 to 150 keV: in diagnostic but also in therapy;
X-Rays (ortho voltage) of 200 to 500 keV which can penetrate a biological tissue thickness of 6 cm;
X-Rays (megavoltage) of 1000 keV to 25 000 keV.

Radioactive isotopes can alternatively be used as ionizing radiation (typically in the context of curie therapy or brachytherapy). In particular, Iodine I-125 (t ½=60.1 days), Palladium Pd-103 (t ½=17 days), Cesium Cs-137, Strontium Sr-89 (t ½=50.5 days), Samarium Sm-153 (t ½=46.3 hours), and Iridium Ir-192, can advantageously be used.

Electron beams may also be used as ionizing radiation and have energy typically comprised between 4 MeV and 25 MeV.

In a particular aspect, a specific monochromatic irradiation source can be used for selectively generating X-rays radiation at an energy close to, or corresponding to, the desired X-ray absorption edge of the high-Z elements of the particles selected for use in the context of the invention.

Preferentially, ionizing radiations are X-rays obtained from Linear Accelerator (LINAC) or are protons.

In the context of the present invention, cells are exposed to ionizing radiation in the context of a radiotherapy regimen which is selected from a conventional fractionation regimen, an hyperfractionation regimen, an (accelerated) hypofractionation regimen and a Stereo Ablative Body radiotherapy (SBAR) regimen.

The radiotherapy protocol for each patient is typically defined by the clinical team according to the patient cancer characteristics, the patient clinical staging, the patient health status, etc. The radiotherapy protocol may therefore correspond to a definitive treatment, a palliative treatment, a concurrent or sequential systemic therapy/radiotherapy, a pre-operative radiotherapy or a post-operative radiotherapy.

In a preferred embodiment, the radiotherapy regimen (also named radiotherapy schedule) is a conventional fractionation comprising or consisting in, typically, from 1.6 Gy up to 2.25 Gy per fraction (for example 1.6, 1.8, 2.0, 2.12, 2.25 Gy per fraction), daily (Monday to Friday, i.e. five consecutive days per week), over 5 weeks up to 7 weeks;
In another embodiment, the radiotherapy regimen is a hyperfractionation comprising or consisting in, typically, 1.2 Gy per fraction, twice daily, over 7 weeks (for example from 79.2 Gy up to 81.6 Gy over 7 weeks, twice daily);
In another embodiment, the radiotherapy regimen is an (accelerated) hypofractionation comprising or consisting in, typically, more than 2.5 Gy per fraction, typically 34 Gy delivered in 10 fractions or 40 Gy delivered in 15 fractions, ideally over 2 weeks up to 4 weeks;
In another embodiment, the radiotherapy regimen is a Stereo Ablative Body Radiotherapy (SBAR) comprising or consisting in, typically, between 45 and 60 Gy delivered in 3 fractions, between 48 and 50 Gy delivered in 4 fractions, between 50 and 55 Gy delivered in 5 fractions, typically over 2 weeks.

It is of relevance to select the nanoparticles of the invention, or a composition comprising said nanoparticles, for use in the above-mentioned clinical context, especially when fractionated radiotherapy is used. This is because, in fractionated radiotherapy, the radioenhancers are repeatedly exposed to ionizing radiation over a period of typically at least 2 weeks, preferably 3, or 4 weeks.

The nanoparticles of the present invention are efficacious and stable to ensure an optimal radioenhancement effect, thus ensuring an optimal benefit/risk ratio for the patient throughout the entire period of RT sessions.

The nanoparticles of the invention are also useful in radio-immunotherapy, whereby the patient with cancer receives radiotherapy and immunotherapy. It has been published that nanoparticles of HfO₂, when exposed to ionizing radiation, act as immunomodulators in the tumor environment (see WO2016189125 and WO 2022096291). The present NPs, when exposed to ionizing radiation, can therefore harness the immune system in a similar manner as described in the latter patent applications.

In an embodiment of the invention, the NPs may be administered in combination with at least one immunotherapeutic agent. The patient may receive the NPs (and radiation) prior to, concomitant with or after receiving immunotherapy.

### Biological Tissue, in Particular Soft Tissue, Visualization

The NPs herein described are, in another preferred aspect herein described, for use in visualizing soft tissue in a mammal, preferably in a human patient, in particular for diagnosis purposes, typically for detecting tumor cells. For example, hafnium oxide, as a radio-opaque material, may be used as a contrast agent for CT scan imaging, thereby enabling the detection of tumor cells. For example, the NPs of the invention, as constituted of a high-Z metal oxide material behaving, as such, as a radio-opaque and as a radio-enhancing material, may be used for detecting a tumor, for post-surgery tumor bed delineation, both for visualizing the tumor bed volume and for enhancing the post-operative radiotherapy that may be delivered to increase the local control over cancer recurrence. Optionally, the NPs of the invention may be embedded into a gel or a hydrogel to favor their deposition in the tumor bed. Suitable gel formulations are described in U.S. Ser. No. 10/588,987B2.

Furthermore, noble metal nanoparticles such as gold are excellent for bioimaging detection because of their tunable photoluminescence, large Stokes shift, low photobleaching and good biocompatibility. When excited by external light (UV-visible or infra-red), excited surface plasmon electrons de-excite/decay through the photothermal effect. The photothermal effect produces thermal contrast upon nonradiative plasmon decay. Gold has been recently used for imaging plasmonic nanoparticles, called photothermal imaging (PTI). In general, PTI requires two beams: a heating beam and a probe beam. Once the LSP of the nanoparticle is excited by the heating beam, the temperature of the nanoparticles and their surroundings increases, resulting in changes in their refractive index. This photothermal effect-induced variation of refractive index can be detected using an additional non-resonant laser source, called a probe beam. In the field of using plasmonic nanoparticles for biomedical imaging, utilization of photoacoustic effect-based imaging modality (i.e., photoacoustic tomography, PAT) as well as direct thermal imaging via a thermographic camera may also be envisaged. Plasmonic nanoparticles may also be used for optical fluorescence or optical coherence tomography (OCT), notably for diagnosis purposes (skin cancer).

### Photothermal Therapy (Activated by Laser and Alternating Magnetic Field)

Combined with radiotherapy (RT), and in view of augmenting cancer cell killing efficacy, NPs of the invention may also be used for photothermal therapy when the second nanoparticles are noble metal NPs.

Indeed, when noble metal NPs are exposed to light (UV-visible or infra-red), free electrons on the nanoparticle surface are excited and conduction-band electrons collectively oscillate at the same frequency, which constitutes the localized surface plasmon resonance (LSPR). During the final relaxation step after localized surface plasmon (LSP) excitation of noble metal nanoparticles, typically excitation by external light (UV-visible or infra-red), the photoexcited energy above the Fermi energy transfers to the metallic lattice through electron-phonon collisions. This relaxation step induces thermal dissipation and releases the thermal energy to the surrounding medium: this phenomenon constitutes the photothermal effect. When excited by a laser beam or a laser pulse, the NPs of the invention may locally increase the temperature of the surrounding cancer cells, leading to tumor cells death.

In the same spirit, NPs of the invention may also be used for magnetic hyperthermia when the second NPs are metal oxide NPs. Indeed, when exposed to low-frequency electromagnetic waves (alternating magnetic field), small (<10 nm) iron oxide NPs begin to rotate due to their magnetic spin rotation, thus locally increasing the temperature of the surrounding cancer cells, leading to tumor cells death.

### Composition

The inventors also describe a composition comprising the inventive NPs (or aggregates thereof) composed of a first nanoparticle of at least one high-Z metal oxide, or mixed high-Z metal oxide, material with Z equal to or above 40, the nanoparticle having a density equal to or above (≥) 7 g/cm³ and below (<) 15 g/cm³, to the surface of which are covalently bound smaller second nanoparticles of at least one noble metal and/or nanoparticles of at least one material that is a Fenton or Fenton-like reaction catalyst preferably chosen from a metal oxide, metal sulfide, metal selenide or metal sulfide/selenide. The composition comprises a) the NPs that are optionally coated with a biocompatible surface coating or aggregates thereof, and b) a pharmaceutically acceptable carrier, vehicle or support.

The pharmaceutical composition herein described is, in a preferred aspect herein described, for use in preventing or treating cancer in a human patient.

In one aspect of the invention, the composition containing the particles is for use in altering or destroying target cells in a mammal, particularly in a human, when said cells are exposed to ionizing radiation.

The composition may be in the form of a solid, liquid (NPs in suspension), aerosol, gel, paste, and the like. Preferred compositions are in a liquid or a gel form. Particularly preferred compositions are in liquid form.

The carrier which is employed can be any classical pharmaceutical support for the skilled person, such for example a saline, isotonic, sterile, buffered solution, or a non-aqueous vehicle solution and the like.

The pharmaceutical composition herein described may comprise a vehicle or support chosen from a liposome, viral vector, viral-like particle, albumin-containing carrier, inorganic polymer and organic polymer known to the skilled person. The vehicle or support may also be any other suitable vehicle or support known to the skilled person.

The composition can also comprise stabilizers, surfactants, polymers and the like. It can be formulated for example as an ampoule, aerosol, bottle, tablet, or capsule, by using techniques of pharmaceutical formulation known by the skilled person.

Generally, the composition, in liquid or gel form, comprises between about 0.05 g/L and about 450 g/L of NPs, for example between about 0.05 g/L and about 250 g/L of NPs, preferably at least about 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 85 g/L, 90 g/L, 95 g/L, 100 g/L, 150 g/L, 200 g/L, 250 g/L, 300 g/L, 350 g/L, or 400 g/L of NPs.

In a particular embodiment, the composition further comprises, or is used in combination with, at least one additional therapeutic agent, for example an immunotherapeutic agent or a cytotoxic agent, for treating cancer.

The concentration of particles in the composition can be measured by the dry extract. A dry extract is ideally measured following a drying step of the suspension comprising the particles in a drying oven.

The pharmaceutical composition herein described is, in another preferred aspect herein described, for use in visualizing a tissue, in particular the soft tissue, of a human patient, for example, for diagnosis purposes since it allows the visualization of tumor cells.

In one aspect of the invention, the composition containing the particles is for use in altering or destroying target cells in a mammal, particularly in a human, when said cells are exposed to ionizing radiation.

The composition may be in the form of a solid, liquid (NPs in suspension), aerosol, gel, paste, and the like. Preferred compositions are in a liquid or a gel form. Particularly preferred compositions are in liquid form.

The examples which follow illustrate the invention without limiting the scope thereof.

### EXAMPLES

### Reference Example 1: Suspension of Sodium Hexametaphosphate Coated Hafnium Oxide Nanoparticles

Hafnium oxide (HfO₂) nanoparticles were synthesized by precipitation of hafnium chloride (HfCl₄) with tetramethyl ammonium hydroxide (TMAOH) at a basic pH. The resulting precipitate was transferred to an autoclave and heated at a temperature between 120° and 300° C. to perform crystallization. After cooling, the suspension was washed with deionized water and acidified. Sodium hexametaphosphate solution was added to the previous suspension and the pH was adjusted between 6.5 and 7.5.

### Example 2: Gold Nanoparticles Synthesis

In a 250 mL volume Erlenmeyer flask, set up in an ice bath, 10 mL of 5 mM HAuCl₄·3H₂O (Gold(III) chloride trihydrate—Sigma Aldrich, USA) were mixed with 25 mL of 10 mM sodium citrate (Sigma Aldrich, USA) and 65 mL of distilled water. When the solution temperature reached 4° C., 1 mL of sodium borohydride, NaBH₄ (0.1 mM) was rapidly introduced under vigorous stirring. The solution turned red immediately.

The nanoparticles' suspension was reconcentrated and washed using an Amicon® Ultra-15 centrifugal filter 30 kDa (60 min at 700 G) at RT (Room Temperature).

The gold content of the suspension was determined using Inductively Coupled Plasma-Optical Emission Spectrometry (ICP-OES).

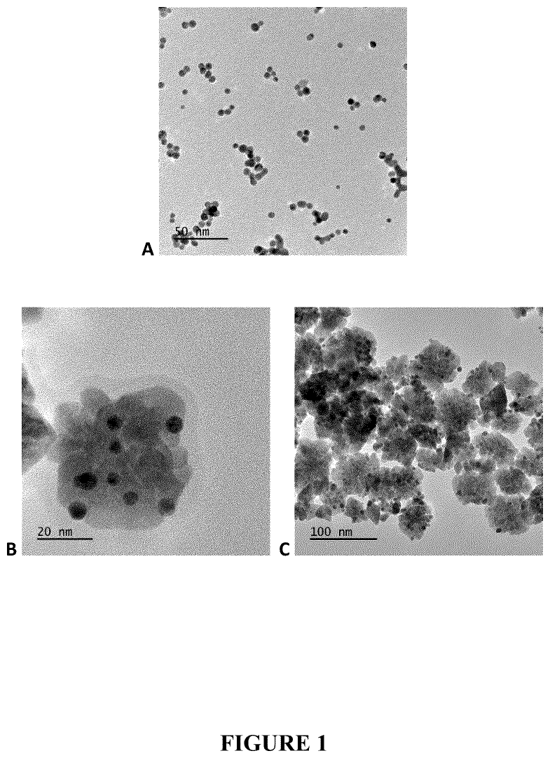

A transmission Electron Microscopy (TEM) micrograph at high resolution was used to visualize the gold nanoparticles (FIG. 1A). A high-resolution electron microscope JEOL 2100 Plus was used for image analysis. The average nanoparticles diameter was calculated over 208 nanoparticles and was found to be 5.7+/−1.1 nm.

The zeta potential was determined by measuring the electrophoretic mobility of the nanoparticles (Zetasizer Nano ZS, Malvern) by diluting the nanoparticles suspension in a NaCl solution at 1 mM at pH 7 (final concentration 0.3 g/L). The zeta potential at pH 7 was found to be −29 mV.

### Example 3. A: HfO₂ Nanoparticle Functionalization (with Amine Group)

Hafnium oxide (HfO₂) nanoparticles were synthesized by precipitation of Hafnium Chloride (HfCl₄) with tetramethyl ammonium hydroxide (TMAOH) at a basic pH. The resulting precipitate was transferred to autoclave at a temperature between 120° C. and 300° C. to perform crystallization. After cooling, the suspension was washed with deionized water and acidified.

3 mL of HfO₂ particle suspension was mixed with 15 mL of absolute ethanol (Fisher Scientific, United Kingdom). The pH was adjusted to 3.2, and (3-Aminopropyl) triethoxysilane (APTES) (Sigma Aldrich, USA) was added to the nanoparticles suspension in a ratio of 1. The resulting suspension was sonicated for 30 minutes and stirred overnight. A white product was collected and washed with ethanol. And then with sterilized water/ethanol v/v(1:1). Finally, the white product was dispersed in filtered water and the pH was adjusted to 7.5.

The hydrodynamic diameter was determined by Dynamic Light Scattering (DLS) with a Zetasizer Nano ZS (Malvern) DLS machine at a scattering angle of 1730 and a laser emitting at 633 nm. The nanoparticles suspension was diluted in water (final concentration 2 g/L). The hydrodynamic diameter of the so obtained nanoparticles in suspension was equal to 52 nm, with a polydispersity index (dispersion of the nanoparticles population in size) of 0.120.

The zeta potential (determined using the conditions described in Example 2, with a final concentration of 2 g/L) was measured at 40 mV.

### Example 3.B: HfO₂ Nanoparticles Decorated with Gold Nanoparticles

5.8 mL of the gold nanoparticles suspension obtained in Example 2 was diluted into 40 mL of sterilized water and the pH was set around 5.8. 150 μL of a solution of 1-Ethyl-3-(3′-dimethylaminopropyl)carbodiimide, HCl (EDAC) (Merck) (6.5 mM) was added dropwise. The pH was adjusted to about 5.8. After stirring, the resulting mixture was washed three times with water and then dispersed in 40 mL of filtered water. The pH was adjusted to about 7.5 and the suspension was kept under stirring at room temperature.

1 mL of the suspension of Example 3.A (diluted at 15 g/L) was then added under stirring. After two days of reaction, the resulting suspension was centrifuged to isolate the obtained nanoparticles and then redispersed in 4 mL of filtered water.

Then, a surface coating step was performed using sodium hexametaphosphate (HMP—Univar, United States). HMP was added to the nanoparticles suspension in a ratio of 0.1 (HMP/HfO₂ w/w) at acidic pH. After stirring, the pH was adjusted around 7 and the solution was washed by centrifugation and dispersed into 2 mL of filtered water.

The hydrodynamic diameter was determined by DLS using the same conditions as those described for Example 3.A, with a final concentration of 0.7 g/L. The hydrodynamic diameter of the so obtained nanoparticles in suspension was equal to 51 nm, with a polydispersity index (dispersion of the nanoparticles population in size) of 0.185.

The zeta potential (determined using the conditions described in Example 2, with a final concentration of 0.7 g/L) at pH 7 was found to be −38 mV.

The hafnium and gold content of the suspension was determined using Inductively Coupled Plasma-Optical Emission Spectrometry (ICP-OES). The ratio Hf/Au was 27 (w/w).

The Transmission Electron Microscopy (TEM) micrograph (FIG. 1B and C) at high resolution confirmed the presence of gold nanoparticles on HfO₂ particles. Jeol 2100 Plus was used for analysis.

### Example 4: Evaluation of Example 3.B Radio Enhancement

The performance of HfO₂ nanoparticles decorated with gold nanoparticles of Example 3.B activated by ionizing radiation was assessed by clonogenic survival assays to define radiosensitivity parameters (SF) and Dose Enhancement Factor (DEF) as described below.

CT26 cells were seeded into 6-well plates (in triplicate) within the range of 100 to 1500 cells/well. The culture medium was RPMI supplemented with 10% FCS and 1% penicillin-streptomycin to facilitate clone formation. HfO₂ concentration of 800 μM was tested in each experiment. Once cells were attached to the plate, each nanoparticles suspension was added overnight (15 h). The cells were irradiated 15-16 h post-treatment with a single dose delivery (0 Gy [sham control], and 4 Gy) using a 160 keV irradiator. The cells were cultured for up to 7 days at 37° C. under 5% CO₂ humidified atmosphere, allowing the cells to form colonies. The colonies were fixed and stained with crystal violet solution and individual colonies were counted to evaluate the plating efficiency and the survival fraction after treatment. Only clones with more than 50 cells were considered viable.

### Data Analysis:

The plating efficiency (PE) which represents the ratio of the number of colonies formed to the number of cells seeded, was calculated as follows for the control and the surviving fraction from each experiment:

$$PE = \frac{n° of colonies formed}{n° of cells seeded} \times 100$$

The surviving fraction (SF) is the number of viable cells after treatment, normalized to the control:

$$SF = \frac{n° of colonies formed after treatment}{n° of cells seeded \times PE}$$

For each experiment, the SF mean from the triplicate was estimated to calculate the enhancement factor (DEF) according to the following formula:

$$DEF ( radiation dose X ) = \frac{mean SF ( radiation dose X )}{mean SF ( radiation dose X + Radioenhancer Product )}$$

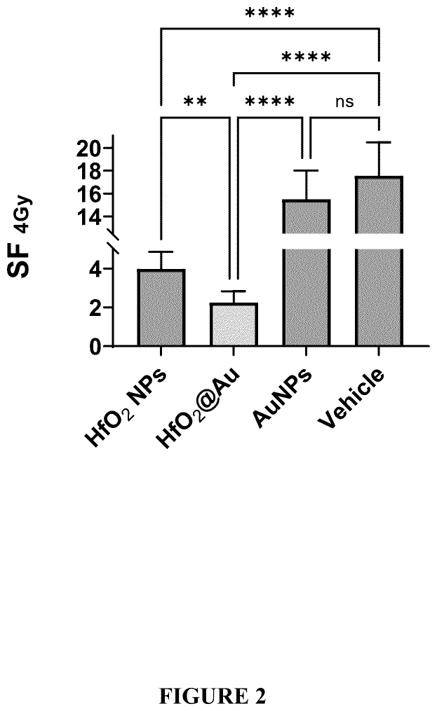

Results: The data are shown in FIG. 2. The Surviving Fractions at 4 Gy (SF_{4Gy}) are summarized in Table 4 below.

TABLE 4

| | HfO₂ NP | HfO₂@Au | Au NP | Vehicle |
|---|---|---|---|---|
| SF 4 Gy Mean (±SD) | 4.0 (±0.68) | 2.3 (±1.70) | 15.5 (±2.82) | 17.9 (±2.11) |

HfO₂ nanoparticles (HfO₂NPs, in FIG. 2) exhibit a radioenhancer effect at a dose of 4 Gy, with an SF of 4.0 (±0.68), compared to 17.9 (±2.11) with radiation only (Vehicle in FIG. 2). Treatment with the inventive NPs according to Example 3B above (HfO₂@Au in FIG. 2) resulted in an SF of 2.3 (±1.70), which is significantly lower compared to the HfO₂ nanoparticle group. The NPs of Example 3B have a significantly higher radioenhancement effect compared to HfO₂ nanoparticles at equal Hf⁺⁴ concentrations. The gold nanoparticles (indicated as Au NPs in FIG. 2) had an SF of 15.5 (±2.82), which is not significantly different from the Vehicle. In this experiment, treatment of the cells with nanoparticles of Example 3B led to a greater than expected radioenhancement effect. Indeed, the nanoparticles of Example 3B showed a synergistic radioenhancer effect that is greater than the combination of the radioenhancer effect observed for the HfO₂ nanoparticles and Au nanoparticles taken separately.

### Example 5: Fe₃O₄ Ultrasmall Nanoparticles Synthesis

The protocol was adapted from Shen (2013, One-step Synthesis of Monodisperse, Water-Soluble Ultra-small Fe₃O₄ Nanoparticles for Potential Bio-application. Nanoscale, 5(5), 2133-2141). 2 mmol of anhydrous FeCl₃ (Fisher Chemical from Fisher BioReagents, USA) were dissolved in 20 mL of diethylene glycol (Fluka, by Sigma Aldrich, Belgium) and 1.3 mmol of Citric acid trisodium salt dihydrate 99% pure (ACROS ORGANIC, by Sigma Aldrich, Belgium) was added under strong stirring. The mixture was then heated at 80° C. under vigorous stirring to form a clear solution. Afterward, 9 mmol of anhydrous sodium acetate (Sigma Aldrich, Germany) was added to the solution under vigorous stirring. The resultant solution was transferred to a Teflon-lined stainless-steel autoclave (25 mL capacity) and placed for 10 hours in a preheated oven at 200° C. After being allowed to cool naturally to room temperature, the black products were collected by centrifugation and washed three times with ethanol. The black powder was redispersed by sonication into 30 mL of sterilized water.

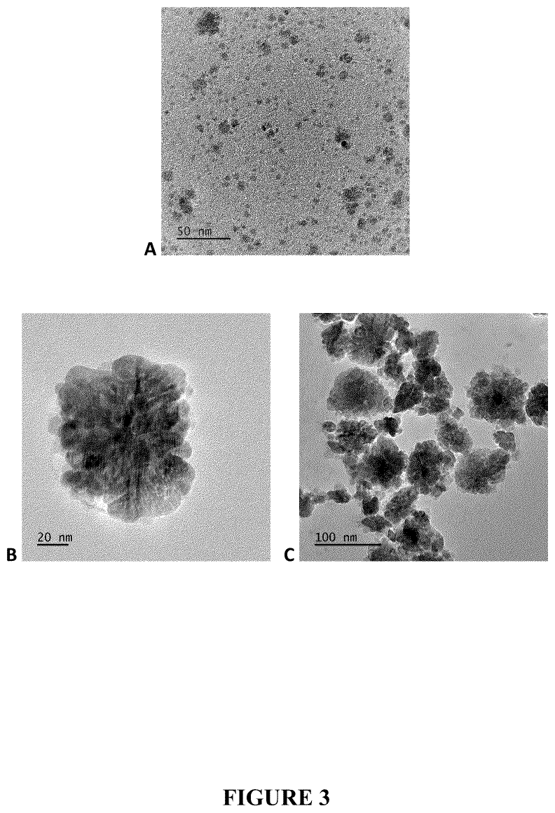

The Transmission Electron Microscopy (TEM) micrograph (FIG. 3A) at high resolution showed the ultrasmall Fe₃O₄ nanoparticles (FIG. 3A). The average diameter was calculated over 300 nanoparticles and was found equal to 5.6+/−2.2 nm.

The zeta potential of the ultrasmall Fe₃O₄ at pH 7 was found equal to −44 mV.

### Example 6: HfO₂ Nanoparticles Decorated with Iron Oxide Nanoparticles

An aqueous suspension of HfO₂ nanoparticles decorated with Fe₃O₄ nanoparticles was obtained by the assembly of both nanoparticles through amide bonding. 8.8 mL of Fe₃O₄ ultrasmall nanoparticles obtained in Example 5 was diluted into 243 mL of sterilized water and pH was adjusted to 5.8. 30 mL of a solution of 1-Ethyl-3-(3′-dimethylaminopropyl)carbodiimide, HCl (EDAC) (Merck) (34 mM) was added dropwise. The pH was adjusted to about 5.8. After more than one hour of stirring, the pH was adjusted again to around 5.8, and stirring was continued for a further hour at RT. The resulting mixture was then washed three times by centrifugation and dispersed using sonication into 250 ml of sterilized water. The pH was then adjusted to 7.5 and the suspension was kept under stirring at RT. 3.6 mL of the suspension from Example 3.A was then added under stirring. After two hours of reaction, the suspension was centrifuged three times at 3000 RPM for 20 minutes and was finally redispersed into 10 mL of sterilized water.

Then, a surface coating step was performed using sodium hexametaphosphate (HMP—Univar, United States). HMP was added to the nanoparticles' suspension in a ratio of 0.03 (HMP/HfO₂ w/w) at acidic pH. The mixture was then sonicated for 5 minutes and centrifuged at 4000 rpm for 20 minutes. The product was finally dispersed into 10 mL of sterilized water and pH was adjusted around 7.

The Transmission Electron Microscopy (TEM) micrograph (FIGS. 3B and 3C) at high resolution was used to visualize HfO₂ nanoparticles decorated with Fe₃O₄ultrasmall nanoparticles.

The hydrodynamic diameter was equal to 77 nm, with a polydispersity index (dispersion of the nanoparticles population in size) of 0.122.

The zeta potential was found to be equal to −30 mV.

The hafnium and iron content of the suspension was determined using Inductively Coupled Plasma-Optical Emission Spectrometry (ICP-OES). The ratio Hf/Fe was 27 (w/w).

### Reference Example 7: Hafnium Oxide First Nanoparticles with Gold Second Nanoparticles Bound at the Surface by Electrostatic Interaction

HAuCl₄ solution (1 g/L, 4.2 mL) (Gold(III) chloride trihydrate—Sigma Aldrich, USA) and polyacrylic acid solution (1 wt %, 0.25 mL) (Sigma Aldrich, USA) were diluted to 62 mL with deionized water under vigorous stirring in the ice bath. After 15 min, NaBH₄ solution (0.1 mol/L, 0.5 mL) was added to the above solution, and a dark brick-red solution was obtained, which indicated that the Au colloids were formed.

A transmission Electron Microscopy (TEM) micrograph at high resolution was used to visualize the gold nanoparticles. The average nanoparticles diameter was calculated over 200 nanoparticles and was found to be 5.3+/−1.3 nm.

Then, a HfO₂ suspension (120 g/L; 0.2 mL) was added. After stirring, the pH was adjusted to 7 and the solution was washed three times by centrifugation and dispersed into filtered water.

The hafnium and gold contents of the suspension was determined using Inductively Coupled Plasma-Optical Emission Spectrometry (ICP-OES). The ratio Hf/Au was 86 (w/w).

The hydrodynamic diameter was determined by DLS using the same conditions as those described for Example 3.A, with a final concentration of 0.1 g/L. The hydrodynamic diameter of the so obtained nanoparticles in suspension was equal to 77 nm, with a polydispersity index (dispersion of the nanoparticles population in size) of 0.114.

The Hf/Au ratio of 86 for this product is too high to obtain the synergistic electronic effect seen for the inventive NPs described in example 3B.

What is claimed is:

1. A nanoparticle (NP) or aggregate of nanoparticles (NPs), wherein the NP is composed of a first nanoparticle of at least one metal oxide (MₓO_{y}) or mixed metal oxide (MₓM′_{z}O_{y}) having an atomic number Z of at least 40, the first nanoparticle having a density equal to or above (≥) 7 g/cm³ and below (<) 15 g/cm³, to which smaller second nanoparticles are covalently bonded at the surface of the first nanoparticle via a linker, wherein the second nanoparticles are composed of either:
a. at least one noble metal chosen from gold (Au), platinum (Pt), Ruthenium (Ru), Rhodium (Rh), palladium (Rd), osmium (Os) and iridium (Ir), and/or
b. at least one metal oxide, metal hydroxide, metal oxy-hydroxide, metal peroxide, metal sulfide, metal selenide, or metal sulfide/selenide that is a Fenton or Fenton-like reaction catalyst, wherein the metal is chosen from the group Fe, Co, Ni, Cu and Mn,
and optionally, a negatively charged or neutrally charged biocompatible surface coating, wherein the first nanoparticle metal content/second nanoparticle metal content ratio is below 30 weight/weight (w/w).

2. The nanoparticle (NP) according to claim 1 wherein the high-Z metal oxide (MₓO_{y}) is HfO₂ or ZrO₂.

3. The nanoparticle (NP) according to claim 1 wherein the second nanoparticles are composed of gold (Au).

4. The nanoparticle (NP) according to claim 1 wherein the second nanoparticles are composed of iron oxide (Fe₃O₄).

5. The nanoparticle (NP) according to claim 1, wherein the first nanoparticle metal content/second nanoparticle metal content ratio is between 20 and 30 weight/weight (w/w).

6. The nanoparticle (NP) according to claim 1, wherein the linker on the first nanoparticle is an amino-silane and the linker on the second nanoparticle is a citrate.

7. The nanoparticle NP according to claim 1, wherein the first nanoparticle has a size of between 40-100 nm.

8. The nanoparticle (NP) according to claim 1, wherein each of the second nanoparticles has a size of between 2-15 nm.

9. A nanoparticle (NP) or an aggregate of nanoparticles (NPs) of claim 1, for use in altering or destroying target tumor cells in a mammal when said cells are exposed to ionizing radiation chosen from X-rays, γ-rays and protons.

10. The nanoparticle (NP) or aggregate for use according to claim 9, wherein cells are exposed to ionizing radiation in the context of a radiotherapy regimen selected from a conventional fractionation regimen, an hyperfractionation regimen, an (accelerated) hypofractionation regimen, and a Stereo Ablative Body radiotherapy (SBAR) regimen.

11. The nanoparticle (NP) or aggregate for use according to claim 9, wherein the target cells are from a solid malignant tumor selected from a skin cancer, a central nervous system cancer, a head and neck cancer, a lung cancer, a breast cancer, a gastrointestinal cancer, a male genitourinary cancer, a gynecologic cancer, an adrenal and/or retroperitoneal cancer, a sarcoma and a pediatric cancer.

12. The nanoparticle (NP) or aggregates of nanoparticles (NPs) of claim 1, for use in visualizing soft tissue and detecting tumor cells in a mammal.

13. A composition comprising a) nanoparticles (NPs) or aggregates thereof as described in claim 1, or their mixtures, and b) a pharmaceutically acceptable carrier, vehicle or support.

14. A composition according to claim 13 for use in altering or destroying target tumor cells in a mammal, particularly in a human, when said cells are exposed to ionizing radiation.

15. A composition according to claim 13 for use in visualizing soft tissue and detecting tumor cells in a mammal, using CT scan.
